(19) United States
(12) Patent Application Publication
(10) Pub. No.: US 2011/0196020 A1
(43) Pub. Date: Aug. 11, 2011

(54) TREATMENT OF CHRONIC FATIGUE SYNDROME USING SELECTIVE AGONISTS OF TOLL-LIKE RECEPTOR 3 (TLR3)

(76) Inventors: William A. Carter, Spring City, PA (US); David Strayer, Bryn Mawr, PA (US)

(21) Appl. No.: 12/998,339

(22) PCT Filed: Oct. 13, 2009
(86) PCT No.: PCT/US2009/005576

§ 371 Date: Apr. 8, 2011

(30) Foreign Application Priority Data
Oct. 10, 2008 (US) 61136889

(51) Int. Cl. A61K 31/713; C07H 21/02; A61P 43/00; A61P 37/00

(52) U.S. Cl. 514/44 R; 536/23.1

(57) ABSTRACT

A subset of human patients having chronic fatigue syndrome and impaired physical performance is treated using one or more different double-stranded ribonucleic acids (dsRNA) or other selective agonists of Toll-like receptor 3 (TLR3).

### CROSS REFERENCE TO RELATED APPLICATIONS

This application claims priority benefit of U.S. provisional application Ser. No. 61/136,889, filed Oct. 10, 2008.

### FIELD OF THE INVENTION

The invention relates to the treatment of a subset of human patients having chronic fatigue syndrome and impaired physical performance using one or more double-stranded ribonucleic acids (dsRNA) or other agonists of Toll-like receptor 3 (TLR3). Medicaments, processes for their manufacture, and methods for their use are provided herein.

### BACKGROUND OF THE INVENTION

Chronic fatigue syndrome is characterized by persistent and disabling fatigue of at least six months duration, which is not explained by another medical condition. See Afari & Buchwald, American Journal of Psychiatry, 160: 221-236 (2003). But the connection between chronic fatigue syndrome and physical performance, if any, was not established: “several studies have focused on chronic fatigue syndrome patients' strength, level of conditioning, and physiological response to exercise, with mixed results.” Id. at 225. The authors conclude, “While these findings do not clarify the role of exercise capacity in chronic fatigue syndrome, they do suggest that the perception of increased effort, decreased activity, and ensuing physical deconditioning can perpetuate the symptoms of chronic fatigue syndrome.” Id. In Table 1, they provide an extensive list of treatment studies, which include using AMPLIGEN® (rintatolimod) poly(I:C₁₂U). A subset of patients, however, effectively treated by dsRNA was not identified. Thus, although Afari and Buchwald suggested that impaired physical performance can perpetuate symptoms of chronic fatigue syndrome, they did not teach or suggest that impaired physical performance identified a treatable subset of patients.

Although the prior art defined patients having chronic fatigue syndrome by behavioral or cognitive criteria, we claim herein that a specifically-configured dsRNA is an effective therapeutic agent for a subset of chronic fatigue syndrome patients selected for impaired physical performance. The effectiveness can be confirmed by improvement of at least one or more physical symptoms.

U.S. Pat. No. 6,130,206, which claims an earliest filing date of Jul. 7, 1980, describes a method of treating a subset of patients suffering from chronic fatigue syndrome with dsRNA. This subset of patients had many different viruses replicating in them. The different viruses included cytomegalovirus, Epstein-Barr virus, other human herpes viruses, and retroviruses. It was discovered that the activity of 2′-5′ oligoadenylate synthetase is abnormally low and ribonuclease (RNase) L acquires aberrant new activities in lymphocytes from the subset of virally-infected patients.

U.S. Pat. No. 5,258,369, which claims an earliest filing date of Aug. 29, 1988, describes a method of treating a subset of patients suffering from chronic fatigue syndrome with dsRNA. This subset of patients had chronic cerebral dysfunction. MRI showed brain abnormalities. They developed a post-infectious immune dysfunction characterized by progressive mental deterioration, memory lapses, occasional seizures, and loss of higher mental abilities (i.e., chronic cerebral dysfunction). It was also discovered that a hyperactive or aberrant 2′-5′ oligoadenylate/RNase L pathway exists in the subset of patients having chronic cerebral dysfunction. Further; natural killer (NK) cell function and NK cell phenotype were often unusual in this subset of patients. The majority of patients were infected by human herpes virus-6 (HHV-6).

Chronic fatigue syndrome patients were treated with AMPLIGEN® (rintatolimod) poly(I:C₁₂U) and had their clinical response measured by Karnofsky performance score, activities of daily living, and exercise treadmill performance. Carter et al., Clinical Infectious Diseases, 18 (suppl. 1): S88-S95 (1994). The authors, however, did not identify a subset of patients having chronic fatigue syndrome that were amenable to treatment because they had impaired physical performance.

In none of the above studies were patients selected for treatment of chronic fatigue syndrome using a clinically measurable criterion such as impaired physical performance (e.g., exercise tolerance testing).

Therefore, it was our objective to identify a different subset of chronic fatigue syndrome patients who are efficaciously treated by dsRNA. This subset of patients is characterized by impaired physical performance. They did not have chronic cerebral dysfunction. Monitoring of treatment efficacy does not rely on normalization of the 2′-5′ oligoadenylate/RNase L pathway. Instead, improvement in at least one or more physical symptoms can be used to assess effectiveness of treatment. Further, instead of assaying lymphocyte or NK cells, the normalization of dendritic cells' function and phenotype is observed. Other advantages and improvements are described below or would be apparent from the disclosure herein.

### SUMMARY OF THE INVENTION

It is an objective of the invention to improve at least one physical symptom or at least the function or phenotype of dendritic cells in a subset of human patients having chronic fatigue syndrome and impaired physical performance using at least one or more different double-stranded ribonucleic acids (dsRNA) or other selective agonists of Toll-like receptor 3 (TLR3).

The effectiveness of treatment may be assessed by measuring the patient's physical performance (e.g., Karnofsky performance score, activities of daily living, exercise tolerance, vitality, or any combination thereof) before, during, and/or after treatment. No cognitive test or measurement of the 2′-5′ oligoadenylate/RNase L pathway is required to assess treatment effectiveness. Virus replication in the patient or infection of the patient does not need to be measured for human herpes viruses (e.g., cytomegalovirus, Epstein-Barr virus, HHV-6).

In one aspect, the at least dsRNA or other TLR3 agonist is administered to a human patient in need of such treatment. A specifically configured or mismatched dsRNA is preferred, but other types of dsRNA may also be used. In particular, the specifically-configured dsRNA is a mismatched dsRNA. The dsRNA may be administered at a dosage of from about 10 to about 1200 mg/dose. This dosage may be administered once per week or month, or two or more doses per week or month. Each dose (e.g., from about 10 mg to about 1200 mg, from about 100 mg to about 800 mg, or from about 200 mg to about 400 mg) may be administered by intravenous infusion. Use of an effective amount of at least dsRNA or TLR3 agonist may be continued until one or more physical symptoms are improved as determined by, for example, Karnofsky performance score (KPF), activities of daily living (ADL), treadmill exercise, vitality, or any combination thereof. The effective amount required to obtain such improvement may be identical to or higher than the amount required for maintenance of the effect(s). For example, physical performance may be assessed before (to establish at least suitability for treatment, baseline performance, or both) and after treatment (to confirm effectiveness of treatment) by at least one or more numerical scores from standardized questions or instruments, treadmill exercise, or both. In particular, a patient with impaired physical performance on a treadmill may be selected by having a cardiac stress test or exercise tolerance test (ETT) value of less than 18 minutes, more preferably ETT less than or equal to nine minutes.

In some aspects, the at least dsRNA or TLR3 agonist is used with the proviso that at least the patient is not cognitively impaired by chronic cerebral dysfunction, cognitive ability of the patient (e.g., intelligence or memory) is not tested, the brain is not scanned by magnetic resonance imaging, the patient is not infected by specific viruses (e.g., CMV, EBV, and/or HHV-6), replication of specific viruses (e.g., CMV, EBV, and/or HHV-6) in the patient is not assayed, a change in the activity of the 2′-5′ oligoadenylate/RNase L pathway in the patient is not measured, or any of the combinations thereof.

The dsRNA may act selectively through a TLR3 receptor. The function and phenotype of dendritic cells may be normalized in the treated patient. This may be used to diagnose a patient as in need of treatment or the efficacy of dsRNA or, alternatively, thereby to improve one or more physical symptoms of a patient afflicted by chronic fatigue syndrome. Use of the dsRNA may correct dendritic cell maturation abnormalities in the patient.

In another aspect, a medicament is provided as a pharmaceutical composition containing one or more different dsRNA or other TLR3 agonists. In particular, the dsRNA may be specifically configured, or more preferably mismatched. Optional components of the composition include excipients and a vehicle (e.g., saline buffer) as a single dose or a multi-dose package (e.g., an injection vial or vials), and instructions for their use. Processes for making and using the pharmaceutical composition (medicament) are also provided. For example, one or more different dsRNA may be formulated at a concentration from about 1 mg/mL to about 5 mg/mL (e.g., 200 mg dissolved in 80 mL or 400 mg dissolved in 160 mL) in physiological phosphate-buffered saline and stored at from 2° C. to 8° C. in a refrigerator under aseptic conditions.

Further aspects of the invention will be apparent from the following description of specific embodiments and the appended claims, and generalizations thereto.

### BRIEF DESCRIPTION OF THE DRAWINGS

FIGS. 1 and 8 show the serum levels of IL-10 (pg/mL) for patients, who were randomly assigned to a group treated with AMPLIGEN® (rintatolimod) or placebo, ranked from lowest to highest value. FIG. 1 shows the baseline values, and FIG. 8 shows the values at week 32 (or last observation). FIG. 15 shows the difference between FIGS. 1 and 8.

FIGS. 2 and 9 show the serum levels of IL-12 (pg/mL) for patients, who were randomly assigned to a group treated with AMPLIGEN® (rintatolimod) or placebo, ranked from lowest to highest value. FIG. 2 shows the baseline values, and FIG. 9 shows the values at week 32 (or last observation). FIG. 16 shows the difference between FIGS. 2 and 9.

FIGS. 3 and 10 show the serum levels of IL-6 (pg/mL) for patients, who were randomly assigned to a group treated with AMPLIGEN® (rintatolimod) or placebo, ranked from lowest to highest value. FIG. 3 shows the baseline values, and FIG. 10 shows the values at week 32 (or last observation). FIG. 17 shows the difference between FIGS. 3 and 10.

FIGS. 4 and 11 show the serum levels of interferon alpha (pg/mL) for patients, who were randomly assigned to a group treated with AMPLIGEN® (rintatolimod) or placebo, ranked from lowest to highest value. FIG. 4 shows the baseline values, and FIG. 11 shows the values at week 32 (or last observation). FIG. 18 shows the difference between FIGS. 4 and 11.

FIGS. 5 and 12 show the serum levels of interferon beta (pg/mL) for patients, who were randomly assigned to a group treated with AMPLIGEN® (rintatolimod) or placebo, ranked from lowest to highest value. FIG. 5 shows the baseline values, and FIG. 12 shows the values at week 32 (or last observation). FIG. 19 shows the difference between FIGS. 5 and 12.

FIGS. 6 and 13 show the serum levels of interferon gamma (pg/mL) for patients, who were randomly assigned to a group treated with AMPLIGEN® (rintatolimod) or placebo, ranked from lowest to highest value. FIG. 6 shows the baseline values, and FIG. 13 shows the values at week 32 (or last observation). FIG. 20 shows the difference between FIGS. 6 and 13.

FIGS. 7 and 14 show the serum levels of tumor necrosis factor (TNF) alpha (pg/mL) for patients, who were randomly assigned to a group treated with AMPLIGEN® (rintatolimod) or placebo, ranked from lowest to highest value. FIG. 7 shows the baseline values, and FIG. 14 shows the values at week 32 (or last observation). FIG. 21 shows the difference between FIGS. 7 and 14.

### DESCRIPTION OF SPECIFIC EMBODIMENTS OF THE INVENTION

Chronic fatigue syndrome is diagnosed by use of criteria from the Center for Disease Control (CDC). See Fukuda et al., Annals of Internal Medicine, 12: 953-959 (1994). In the context of the present invention, it would not be necessary to consider impairment of short-term memory one of the criteria. Patients may be selected for treatment based on impaired physical performance (preferably exercise tolerance testing on a treadmill) and having a longer duration of physical debilitation (i.e., much more than six consecutive months) than required by the CDC criteria.

Physical performance of a human patient having chronic fatigue syndrome can be assessed by at: least the patient's Karnofsky performance score (KPS), activities of daily living (ADL), treadmill exercise, vitality, or a combination thereof.

KPS can be measured by a physician's assessment of the patient's function based on a structured interview, direct observation, discussion of any specific signs and symptoms, and basic functional accomplishments.
100—normal, no complaints, no signs of disease
90—capable of normal activity, few symptoms or signs of disease
80—normal activity with some difficulty, some symptoms or signs
70—caring for self, not capable of normal activity or work
60—requiring some help, can take care of most personal requirements
50—requires help often, requires frequent medical care
40—disabled, requires special care and help
30—severely disabled, hospital admission indicated but no risk of death
20—very ill, urgently requiring admission, requires supportive measures or treatment
10—moribund, rapidly progressive fatal disease processes
0—death
   Alternatively, performance scoring can be measured using modifications of KPS such as those by David Bell (The Doctor's Guide to Chronic Fatigue Syndrome, Da Capo Press, 1995) and Charles Shepherd (Living with M.E., 3^{rd} Ed., Random House UK, 1999). Weekly KPS scores may be averaged for each patient under treatment over four-week intervals of treatment. The maximum score is 100 for an asymptomatic person. Within the range of from 40 to 80, changes of 10 are clinically meaningful.

The Barthel ADL index can be modified and used for scoring performance of 83 discrete activities of daily living by self-assessment in 13 modules (i.e., bathing, housekeeping, communication, dressing, grooming, home management, laundry, meal preparation, mobility/activity, physical manipulation, vehicular transportation, toilet, and yard work/maintenance). See Mahoney & Barthel, Maryland State Medical Journal, 14: 61-65 (1965) and Collin et al., International Disability Studies, 10: 61-63 (1988). Weekly ADP values may be averaged for each patient under treatment over four-week intervals of treatment. The score is calculated by averaging scores in each module, then averaging scores of the 13 modules and multiplying by 20. There are five levels of performance (i.e., 1=unable to do, 2=need help most of the time, 3=need help some of the time, 4=no help needed but symptoms present, and 5=no help needed and no symptoms). The scale ranges from 20 to 100; the maximum score is also 100 for an asymptomatic person. For alternative indices of scoring ADL, see McDowell, Measuring Health, 3^{rd} Ed., Oxford Univ. Press (2006).

Exercise on a treadmill can measure exercise capacity, tolerance, recovery, or a combination thereof. The duration of exercise tolerance may be used as a measure of treatment efficacy because it is a frequently used endpoint to support efficacy of therapeutic agents used to treat patients with debilitating chronic diseases. The primary limitation experienced by patients having chronic fatigue syndrome is an inability to engage in physical activity for extended periods (e.g., ETT less than 18 minutes, preferably ETT less than or equal to nine minutes). A placebo-adjusted increase in mean treadmill exercise duration from baseline of at least 6.5% is clinically meaningful.

The Short Form-36 (SF-36) health survey is self-administered by the patient to assess subjective well-being related to nine health concepts. The subscale of interest for vitality scoring is the Vitality Index. This comprises four items in Section 9 of SF-36 dealing with the amount of time in the past four-week interval that the patient felt full of pep, had a lot of energy, felt worn out, and felt tired.

The subset of human patients in need of treatment having impaired physical performance may be selected by a reduced quality of life as determined by Karnofsky performance score (KPS). For example, the patient is determined to have a KPS of 40 to 60 on three occasions, each at least 14 days apart, during the 12 weeks prior to treatment. Concomitantly or alternatively, the patient is determined to have an ETT of less than 18 minutes, preferably less than or equal to nine minutes.

The double-stranded ribonucleic acid (dsRNA) may be fully hybridized strands of poly(riboinosinic acid) and poly(ribocytidilic acid) (i.e., polyIC) or poly(riboadenylic acid) and poly(ribouracilic acid) (i.e., polyAU). If mismatched, the dsRNA may be of the general formula rIₙ·r(C₄₋₂₉U)ₙ, which is preferably rIₙ·r(C₁₂U)ₙ, in which r indicates ribonucleotides. It is preferred that n is an integer from about 40 to about 40,000. For example, a strand of poly(riboinosinic acid) may be partially hybridized to a strand of poly(ribocytosinic₄₋₂₉uracilic acid). Other mismatched dsRNA that may be used are based on copolynucleotides such as poly(CₘU) and poly(CₘG) in which m is an integer from about 4 to about 29 or analogs of a complex of poly(riboinosinic acid) and poly(ribocytidilic acid) formed by modifying the rIₙ·rCₙ to incorporate unpaired bases (uracil or guanine) in the polyribocytidylate (rCₘ) strand. Alternatively, mismatched dsRNA may be derived from r(I)·r(C) dsRNA by modifying the ribosyl backbone of poly(riboinosinic acid) (rIₙ), e.g., by including 2′-O-methyl ribosyl residues. Of these mismatched dsRNA analogs of rIₙ·rCₙ, the preferred ones are of the general formula rIₙ·r(C₁₁₋₁₄U)ₙ or rIₙ·r(C₂₉,G)ₙ (see U.S. Pat. Nos. 4,024,222 and 4,130,641; which are incorporated by reference). The dsRNA described therein generally are suitable for use according to the present invention. See also U.S. Pat. No. 5,258,369. The dsRNA may be complexed with an RNA-stabilizing polymer such as polylysine, polylysine plus carboxy-methylcellulose, polyarginine, polyarginine plus carboxymethylcellulose, or any combination thereof.

Other examples of mismatched dsRNA for use in the invention include:

r(I)·r(C₄,U),

r(I)·r(C₇,U),

r(I)·r(C₁₃,U),

r(I)·r(C₂₂,U),

r(I)·r(C₂₀,G) and

r(I)·r(C₂₉,G).

Mismatched dsRNA may also be modified at the molecule's ends to add a hinge(s) to prevent slippage of the base pairs, thereby conferring a specific bioactivity in specific solvents or aqueous environments which exist in human biological fluids.

dsRNA or TLR3 agonist may be administered to a human patient by any local or systemic route known in the art including enteral (e.g., oral, feeding tube, enema), topical (e.g., device such as a nebulizer for inhalation through the respiratory system, skin patch acting epicutaneously or transdermally, suppository acting in the rectum or vagina), and parenteral (e.g., subcutaneous, intravenous, intramuscular, intradermal, or intraperitoneal injection; buccal, sublingual, or transmucosal; inhalation or instillation intranasally or intratracheally). dsRNA or TLR3 agonist may be micronized by milling or grinding solid material, dissolved in a vehicle (e.g., sterile buffered saline or water) for injection or instillation (e.g., spray), topically applied, or encapsulated in a liposome or other carrier for targeted delivery. Preferred are carriers that target the dsRNA or TLR3 agonist to the TLR3 receptor on antigen presenting cells and epithelium. For example, immature dendritic cells may be contacted in skin, mucosa, or lymphoid tissues. It will be appreciated that the preferred route may vary with the age, condition, or gender of the patient; the nature of disease, including the number and severity of symptoms; and the chosen active ingredient.

Formulations for administration (i.e., pharmaceutical compositions) may include aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents. It will be appreciated that the preferred formulation may vary with the age, condition, or gender of the patient; the nature of disease, including the number and severity of symptoms; and the chosen active ingredient.

The recommended dosage of dsRNA or TLR3 agonist will depend on the clinical status of the patient and the physician's experience treating chronic fatigue syndrome. dsRNA may be dosed at from about 10 mg to about 1200 mg, from about 100 mg to about 800 mg, or from about 200 mg to about 400 mg in a patient (e.g., body mass of about 70 kg) on a schedule of once to thrice weekly (preferably twice weekly), albeit the dose amount and/or frequency may be varied by the physician in response to the patient's condition. TLR3 agonist may be dosed in amounts that achieve therapeutically equivalent effects as poly(I:C₁₂U) dosed at the above amounts. Intravenous infusion of dsRNA or TLR3 agonist dissolved in a physiological phosphate-buffered saline is preferred. Cells or tissues that express TLR3 are preferred sites in the patient for delivering the nucleic acid, especially antigen presenting cells (e.g., dendritic cells and macrophages) and endothelium (e.g., endothelial cells of the respiratory and gastric systems). It will be appreciated that the preferred dosage may vary with the age, condition, or gender of the patient; the nature of disease, including the number and severity of symptoms; and the chosen active ingredient.

Dendritic cells which act as sentinel cells possess molecular surface structures that recognize pathogen-associated molecular patterns (PAMPs). These PAMPs include a set of Toll-like receptors (TLRs) that specifically recognize double-stranded RNAs. This TLR is known as Toll-like receptor 3 (TLR3). Poly(I:C₁₂U) is a selective agent for activation of TLR3, but other selective agents known in the art may be used. Dysfunction in co-stimulatory molecule (e.g., CD80, CD83, CD86) signaling in dendritic cells may be associated with the physical symptoms of chronic fatigue syndrome. This abnormality may be normalized by using poly(I:C₁₂U) or another selective agent as a specific agonist of TLR3.

### EXAMPLES

A prospective, double-blind, randomized, placebo-controlled study evaluated the safety and the efficacy of AMPLIGEN® (rintatolimod) that was administered to 234 patients having severe chronic fatigue syndrome. They were randomized into two groups of 117 patients each. Following twice-weekly, intravenous (IV) infusions for two weeks of 200 mg poly(I:C₁₂U) in 80 mL or an equal volume of placebo, 400 mg poly(I:C₁₂U) in 160 mL or an equal volume of placebo was infused IV twice weekly for a total of 40 weeks (Stage 1). At the conclusion of Stage 1, blinding was continued and the placebo-treated patients were crossed-over to poly(I:C₁₂U) treatment, while the original poly(I:C₁₂U) cohort had their treatment continued for another 24 weeks (Stage 2). The two study cohorts were well-balanced with regard to age, gender, ethnicity, and duration of symptoms. A total of 194 patients completed all 40 weeks of Stage 1. In addition, seven patients from each cohort who discontinued the study following initial dosing completed an on-study treadmill test and were included in the statistical analysis.

The poly(I:C₁₂U) was manufactured in accordance with Good Manufacturing Practice (GMP) and tested under Good Laboratory Practice (GLP) and Good Clinical Practice (GCP) guidelines. Randomization schedules of patients were provided by Simirex, Mt. Laurel, N.J. USA. Knowledge of randomization schedules and patient assignments was strictly limited to staff members who had a need to know the randomization code to prepare and package the medicament used in the study.

Exercise tolerance testing was performed by having patients evaluated on a treadmill through as many as 12 programmed stages of increased walking rates and/or inclinations. Each stage, except the final stage, lasted for two minutes. Stage 1 was conducted at 2 miles per hour (mph) with no elevation; stages 2 through 8 at 2 mph, beginning with no elevation and proceeding to 21% elevation (i.e., an increase of 3% elevation per stage); and stages 9 through 12 at 3 mph, 4 mph, 5 mph, and 5 mph, respectively, with 21% elevation at all stages. If is was reached, stage 12 was continued at 5 mph with 21% elevation until termination of the test. Patients progressed through stages successively until they chose to stop.

Exercise tolerance testing was scored as the total time on the treadmill. Two tests were performed to establish baseline physical performance. If the two baseline tests differed in their maximum duration by more than 10% from their mean value, a third test was performed and the two closest times were used for data analysis of ET. In order to reduce variation in test results, each site used the same make and model of treadmill (Trackmaster TM 225, Full Vision, Newton, Kans. USA) and the same group of exercise physiology specialist (The Workwell Physiology Services, Ripon, Calif. USA) traveled to each site to administer the treadmill test throughout the study. Treadmills were calibrated on the day of each test for speed and inclination.

Results from the analysis of covariance (baseline ET) revealed a statistically significant improvement in ET at 40 weeks in favor of patients randomized to receive poly(I:C₁₂U) compared to placebo. Two-sample t-test was used to compare baseline ET between the two randomized study groups. The intra-group changes in ET from baseline to week 40 were analyzed using a paired-difference t-test. The proportion of patients who achieved a 25% or 50% increase in ET at week 40 was compared between randomized study groups using a two-tailed Fisher's exact test. Secondary endpoints were analyzed based on the distribution of the dependent variable (i.e., categorical, continuous, and counts).

An analysis of the primary endpoint, treadmill ET, at 40 weeks in the intent-to-treat (ITT) group is shown in Table 1A. Poly(I:C₁₂U) treatment (n=100) increased mean ET by 96 sec to 672 sec for a 16.6% increase in mean ET. In contrast, the placebo cohort (n=108) increased mean ET by 28 sec (4.8%) to 616 sec for a placebo-adjusted increase of 11.8% in the poly(I:C₁₂U)-treated ITT cohort. For patients who completed all 40 weeks of the study (n=194), mean baseline ET was 583 sec for the poly(I:C₁₂U) cohort (n=93) compared to 587 sec for the placebo cohort (n=101). At week 40, the poly(I:C₁₂U) cohort had increased mean ET by 108 sec (18.6%) to 691 sec compared to an increase of 27 sec (4.6%) to 614 sec in the placebo cohort for a placebo-adjusted increase of 14.0%.

At 40 weeks, the difference in improvement in mean ET for poly(I:C₁₂U) versus placebo for both the completer and ITT groups was statistically significant (p=0.021 and 0.043, respectively) using an analysis of covariance model. A paired-difference t-test for analysis of the intra-patient difference from baseline provided additional evidence that poly(I:C₁₂U) produced a significant increase in mean ET for patients severely debilitated with chronic fatigue syndrome. Both the completer and ITT populations improved mean ET significantly (p<0.001) compared to the placebo cohorts (p>0.19).

TABLE 1
Analysis of the effect of Poly I:Poly C₁₂U on the primary endpoint,
exercise tolerance (ET)
A: Increase in Exercise Treadmill Duration with Poly I:Poly C₁₂U in
CFS Patients (Intent-to-Treat)

| | Mean Exercise | | Percent Increase | | |
|---|---|---|---|---|---|
| | Duration (Seconds) | | from Baseline | | |
| | Poly I: | | Poly I: | | |
| Study | Poly C₁₂U | Placebo | Poly C₁₂U | Placebo | |
| Interval | (n = 100) | (n = 108) | (n = 100) | (n = 108) | p-value |
| Baseline | 576 | 588 | — | — | 0.729* |
| Week 40 | 672 | 616 | 16.6 | 4.8 | 0.043** |
| p-value*** | | | <0.001 | 0.198 | |

B: Increase in Exercise Treadmill Duration with Poly I:Poly C₁₂U in
CFS Patients without Significant Dose Reductions (Intent-to-Treat)

| | Mean Exercise | | Percent Increase | | |
|---|---|---|---|---|---|
| | Duration (Seconds) | | from Baseline | | |
| | Poly I: | | Poly I: | | |
| Study | Poly C₁₂U | Placebo | Poly C₁₂U | Placebo | |
| Interval | (n = 83) | (n = 98) | (n = 83) | (n = 98) | p-value |
| Baseline | 581 | 590 | — | — | 0.813* |
| Week 40 | 690 | 616 | 18.7 | 4.5 | 0.022** |
| p-value*** | | | <0.001 | 0.263 | |

C: Frequency Distribution of Percent Change from Mean Baseline
Exercise Treadmill Duration at Week 40 (Intent-to-Treat)

| Improvement from | | | |
|---|---|---|---|
| Mean Baseline | | | |
| Exercise Treadmill | Poly I:Poly C₁₂U | Placebo | |
| Duration | (n = 100) | (n = 108) | p-value**** |
| At least 25%, n (%) | 39 (39) | 25 (23) | 0.016 |
| At least 50%, n (%) | 26 (26) | 15 (14) | 0.036 |

D: Effect of Baseline ET on Week 40 ET (Intent-to-Treat)

| Base- | Exercise Duration mean (seconds) | | | | | | | |
|---|---|---|---|---|---|---|---|---|
| line | ≦9 | | >9 | | % Gain | | | |
| ET | Poly I: | | Poly I: | | Poly I: | | | |
| Strata | Poly | ≦9 | Poly | >9 | Poly C₁₂U | | | |
| (Min- | C₁₂U | Placebo | C₁₂U | Placebo | over Placebo | | p-value** | |
| utes) | (n = 40) | (n = 42) | (n = 60) | (n = 66) | ≦9 | >9 | ≦9 | >9 |
| Base- | 321 | 353 | 747 | 738 | | | | |
| line | | | | | | | | |
| Week | 450 | 446 | 820 | 725 | 13.9 | 11.6 | 0.517 | 0.034 |
| 40 | | | | | | | | |

*Student's t-test comparing mean baseline ET between treatment groups.
**Analysis of covariance (ANCOVA) comparing the mean ET change from baseline within each treatment group.
***Student's t-test comparing whether the change from baseline is equal to zero within each treatment group.
****Probability that a difference between treatment groups exists using the Fisher exact test.

The effect of dose modification was analyzed by exclusion of patients in the ITT population with significant dose reductions, defined as a combined total of 20 missed doses or dose reductions of at least 50%. Table 1B demonstrates that when patients with significant dose reductions were excluded, the placebo-adjusted mean improvement was 14.3% (p=0.022).

Additional evidence supporting the efficacy of poly(I:C₁₂U) in chronic fatigue syndrome was provided by an analysis of the frequency distribution of percent improvement in ET from baseline to week 40 in the poly(I:C₁₂U) versus placebo cohorts (Table 1C). The proportions of patients in the ITT population with changes in ET from baseline to week 40 of at least 25% or of at least 50% were 1.7-fold or 1.9-fold greater, respectively, for patients randomized to poly(I:C₁₂U) than placebo, 39% versus 23% and 26% versus 14%, respectively (p=0.036).

Sub-group analyses of patients with high baseline ET (greater than 9 minutes at baseline) and low treadmill ET (less than or equal to 9 minutes) provide further insight on the outcome of patients treated with poly(I:C₁₂U). Table 1D demonstrates a clinically significant placebo-adjusted enhancement of treadmill ET of 13.9% in the patients stratified to the low-performance cohort (n=82). In contrast, the majority of the ITT study patients (n=126) were in the high-performance cohort. An 11.6% improvement in ET was seen as a function of treatment with poly(I:C₁₂U) compared to placebo (p=0.034). Stage 2 analysis supports the conclusions from Stage 1. Following the cross-over to Stage 2, the original placebo cohort in a blinded cross-over to poly(I:C₁₂U) treatment achieved a mean intra-patient improvement in ET of 39% (p=0.040) in 24 weeks, while the original poly(I:C₁₂U) cohort maintained their improvement in ET.

Statistically significant changes (p<0.01) in secondary endpoints of physical performance from baseline were observed in KPS, ADL, and vitality scores for patients receiving poly(I:C₁₂U) during Stage 1 (Table 2). Although the placebo cohort did not have a change in median KPS score as a group, the Wilcoxon signed rank analysis indicated a significant shift in the distribution of individual scores. No other secondary endpoints provided evidence of a statistically significant improvement in Stage 1. During the 24 weeks of Stage 2, additional support for the efficacy of poly(I:C₁₂U) was demonstrated. Analysis of the ITT cohort showed that median KPS increased from 50 to 60 (p<0.001) and median ADL increased from 71.4 to 71.7 (p=0.01). No statistically significant change in vitality score was seen during Stage 2.

TABLE 2
Secondary Performance Endpoint Improvements

| Secondary | Poly(I:C₁₂U) | | | Placebo | | |
|---|---|---|---|---|---|---|
| Endpoint | Base- | | | Base- | | |
| Performance | line | Week 40 | p-value | line | Week 40 | p-value |
| Karnofsky | 50 | 55 | <0.01 | 50 | 50 | <0.01 |
| Performance | | | | | | |
| Score (KPS) | | | | | | |
| Activities of | 68.1 | 72.4 | <0.01 | 68.7 | 69.4 | ns |
| Daily Living | | | | | | |
| (ADL) Score | | | | | | |
| Vitality and | 5.0 | 10.0 | <0.01 | 10.0 | 10.0 | ns |
| General Health | | | | | | |
| (SF-36) Score | | | | | | |

In summary, patients receiving poly(I:C₁₂U) in Stage 1 experienced a placebo-adjusted average improvement in ET of 11.8% (p=0.043) from baseline using an intention-to-treat analysis. Correction for patients with reduced dosing compliance increased the placebo-adjusted improvement in ET to 14.3% (p=0.022). This improvement in ET is about a two-fold increase in the minimum threshold considered clinically significant (i.e., 6.5%). A significantly greater proportion of patients receiving poly(I:C₁₂U) also reduced their dependence on other drugs to treat the symptoms of chronic fatigue syndrome (p=0.048) compared to patients randomized to placebo. After 40 weeks, placebo patients who were crossed-over to receive poly(I:C₁₂U) demonstrated an intra-patient improvement in ET of 39% (p=0.04) compared to their original baseline ET.

### Dendritic Cell Maturation Markers

Expression of CD80, CD83, and CD86 was analyzed by flow cytometry using fluorescently-labeled antibodies. Following overnight shipment, blood samples were stained within one hour of receipt. Standard flow cytometry methods were employed for cell marker analyses and lysis of red blood cells. Dendritic cells were identified based on low level expression of monocyte, lymphocyte, and NK cell markers along with high HLA-DR expression. Dendritic cells were also characterized according to CD11c and CD123 expression. Monocytes were identified by side scatter analysis and expression of a monocyte lineage marker. Analyses of CD80, CD83, and CD86 expression were performed after cell type identification. Measurements from healthy volunteers served as controls and indicated normal distribution and levels of marker expression for mature DC such as CD80, CD83, and CD86.

Results of DC maturation marker analyses are reported as percentage of positive-staining cells and by expression level (mean fluorescence intensity, MFI) and are presented as mean (SD) unless otherwise indicated. Data for healthy volunteers not treated with poly(I:C₁₂U) are reported for comparison. CD80, CD83, and CD86 results on a per individual basis are presented below.

Mean (SD) percentages of CD123⁺ DC, CD11⁺ DC, and monocytes are presented in Table 3. Values for healthy volunteers were included as normal values (see Table 4). Healthy volunteers did not receive poly(I:C₁₂U) infusion. Values for patients having chronic fatigue syndrome are reported for specified time points relative to poly(I:C₁₂U) infusion. Mean values were calculated over all measurements for all patients at each time point.

Pre-infusion values for the patients were comparable with healthy volunteers' levels; the percentage of CD11⁺ cells was at the low end of the range for healthy volunteers as defined by the mean and SD. Mean values were below those measured for healthy volunteers for CD123⁺ cells four hours post-infusion and for CD11⁺ cells and monocytes 24 hours post-infusion. One consistent change was a decrease in the percentage of monocytes demonstrated by patients 24 hours post-infusion. Monocyte numbers recovered by 72 hours post infusion (see Table 5). Overall, percentages of CD123⁺ cells, CD11⁺ cells, and monocytes (mono) were slightly low, but not out of the range of values for healthy volunteers.

In general, treatment with poly(I:C₁₂U) decreased the percentage of cells expressing the mature DC markers CD80, CD83, and CD86, and it increased their expression levels. The patients tended to start with more positive cells having lower expression levels than healthy volunteers who received no poly(I:C₁₂U). Thus, the ability of poly(I:C₁₂U) to decrease the number of positive cells and to increase the expression levels of DC maturation markers normalized the patients' profiles such that they more closely resembled those of healthy volunteers.

TABLE 3
Poly(I: C₁₂U) Effects on Cell Populations

| | Number of Cells (% of Leukocytes) | | |
|---|---|---|---|
| | CD123⁺ | CD11⁺ | Monocytes |
| Healthy | 0.17 (0.06) | 0.27 (0.11) | 6.10 (1.12) |
| Volunteers^{a} (n = 6) | | | |

Patients (n = 4)

| | | | |
|---|---|---|---|
| Pre-Infusion | 0.15 (0.09) | 0.12 (0.04) | 4.77 (0.77) |
| 4 hr Post-Infusion | 0.07 (0.03) | 0.17 (0.13) | 4.40 (1.10) |
| 24 hr Post-Infusion | 0.12 (0.03) | 0.08 (0.03) | 3.01 (0.89) |
| 72 hr Post-Infusion | 0.13 (0.05) | 0.19 (0.06) | 4.95 (0.48) |

^{a}Healthy volunteers received no poly(I: C₁₂U) infusions

TABLE 4
Individual Results for Dendritic Cell
Type Percentages in Healthy Volunteers

| Healthy Volunteer | CD123⁺ | CD11c⁺ | Monocytes |
|---|---|---|---|
| 1 | 0.21 | 0.33 | 4.93 |
| | 0.23 | 0.32 | 4.87 |
| | 0.22 | 0.35 | 4.93 |
| | 0.2 | 0.31 | 4.58 |
| Mean | 0.22 | 0.33 | 4.83 |
| SD | 0.01 | 0.02 | 0.17 |
| 2 | 0.14 | 0.19 | 5.02 |
| | 0.14 | 0.16 | 4.92 |
| | 0.15 | 0.16 | 4.82 |
| | 0.15 | 0.15 | 5.01 |
| Mean | 0.15 | 0.17 | 4.94 |
| SD | 0.01 | 0.02 | 0.09 |
| 3 | 0.12 | 0.22 | 5.72 |
| | 0.13 | 0.21 | 5.3 |
| | 0.12 | 0.19 | 5.3 |
| | 0.13 | 0.22 | 5.52 |
| Mean | 0.13 | 0.21 | 5.46 |
| SD | 0.01 | 0.01 | 0.20 |
| 4 | 0.08 | 0.2 | 6.27 |
| | 0.08 | 0.19 | 6.62 |
| | 0.09 | 0.23 | 6.67 |
| | 0.09 | 0.19 | 6.87 |
| Mean | 0.09 | 0.20 | 6.61 |
| SD | 0.01 | 0.02 | 0.25 |
| 5 | 0.19 | 0.23 | 7.53 |
| | 0.18 | 0.24 | 7.71 |
| | 0.18 | 0.23 | 7.67 |
| | 0.18 | 0.23 | 7.67 |
| Mean | 0.18 | 0.23 | 7.65 |
| SD | 0.01 | 0.00 | 0.08 |
| 6 | 0.26 | 0.49 | 7.26 |
| | 0.23 | 0.5 | 7.02 |
| | 0.24 | 0.49 | 7.11 |
| | 0.26 | 0.49 | 7.14 |
| Mean | 0.25 | 0.49 | 7.13 |
| SD | 0.02 | 0.01 | 0.10 |

TABLE 5
Individual Results for Dendritic Cell Type Percentages in Patients

| | Day 1 Pre-Infusion | | | Day 1 Post-Infusion | | | 24 Hours Post-Infusion | | | 72 Hours Post-Infusion | | |
|---|---|---|---|---|---|---|---|---|---|---|---|---|
| Patient ID | CD123⁺ | CD11c⁺ | mono | CD123⁺ | CD11c⁺ | mono | CD123⁺ | CD11c⁺ | mono | CD123⁺ | CD11c⁺ | mono |
| LDM-010 | 0.11 | 0.07 | 3.81 | 0.07 | 0.18 | 3.87 | 0.08 | 0.12 | 2.3 | 0.13 | 0.27 | 5.69 |
| | 0.14 | 0.08 | 4.82 | 0.06 | 0.21 | 3.22 | 0.08 | 0.14 | 2.12 | 0.11 | 0.29 | 5.56 |
| | 0.11 | 0.1 | 3.2 | 0.07 | 0.21 | 3.37 | 0.1 | 0.11 | 2.06 | 0.13 | 0.27 | 5.72 |
| | 0.13 | 0.08 | 3.75 | 0.08 | 0.23 | 3.21 | 0.11 | 0.09 | 2.39 | 0.12 | 0.28 | 5.47 |
| Mean | 0.12 | 0.08 | 3.90 | 0.07 | 0.21 | 3.42 | 0.09 | 0.12 | 2.22 | 0.12 | 0.28 | 5.61 |
| SD | 0.02 | 0.01 | 0.68 | 0.01 | 0.02 | 0.31 | 0.02 | 0.02 | 0.15 | 0.01 | 0.01 | 0.12 |
| JOG-020 | 0.09 | 0.11 | 6.35 | 0.08 | 0.1 | 4.84 | 0.08 | 0.07 | 3.25 | 0.08 | 0.13 | 4.85 |
| | 0.1 | 0.11 | 4.75 | 0.08 | 0.11 | 4.78 | 0.08 | 0.08 | 3.49 | 0.09 | 0.14 | 4.94 |
| | 0.12 | 0.13 | 4.96 | 0.1 | 0.1 | 4.69 | 0.08 | 0.07 | 3.09 | 0.08 | 0.15 | 4.97 |
| | 0.11 | | 4.28 | 0.1 | 0.11 | 4.69 | 0.09 | 0.08 | 3.04 | 0.1 | 0.12 | 4.75 |
| Mean | 0.11 | 0.12 | 5.09 | 0.09 | 0.11 | 4.75 | 0.08 | 0.08 | 3.22 | 0.09 | 0.14 | 4.88 |
| SD | 0.01 | 0.01 | 0.89 | 0.01 | 0.01 | 0.07 | 0.01 | 0.01 | 0.20 | 0.01 | 0.01 | 0.10 |
| JLC-109 | 0.09 | 0.11 | 5 | 0.13 | 0.41 | 6.63 | 0.13 | 0.06 | 5.45 | 0.1 | 0.16 | 4.43 |
| | 0.06 | 0.12 | 4.68 | 0.12 | 0.35 | 4.92 | 0.13 | 0.08 | 3.66 | 0.1 | 0.17 | 4.36 |
| | 0.09 | 0.13 | 4.56 | 0.06 | 0.41 | 5.77 | 0.15 | 0.14 | 2.21 | 0.12 | 0.18 | 4.37 |
| | 0.09 | 0.13 | 4.53 | 0.1 | 0.27 | 6.2 | 0.14 | 0.09 | 1.8 | 0.11 | 0.17 | 4.21 |
| Mean | 0.08 | 0.12 | 4.69 | 0.10 | 0.36 | 5.88 | 0.14 | 0.09 | 3.28 | 0.11 | 0.17 | 4.34 |
| SD | 0.02 | 0.01 | 0.22 | 0.03 | 0.07 | 0.73 | 0.01 | 0.03 | 1.65 | 0.01 | 0.01 | 0.09 |
| DMM-111 | 0.33 | 0.1 | 5.45 | 0.03 | 0.02 | 3.94 | 0.13 | 0.04 | 3.44 | 0.2 | 0.18 | 4.82 |
| | 0.33 | 0.11 | 5.26 | 0.03 | 0.02 | 3.44 | 0.14 | 0.03 | 3.26 | 0.21 | 0.17 | 4.98 |
| | 0.32 | 0.22 | 5.48 | 0.03 | 0.02 | 3.39 | 0.16 | 0.05 | 3.6 | 0.21 | 0.16 | 4.85 |
| | 0.24 | 0.16 | 5.42 | 0.03 | 0.02 | 3.49 | 0.16 | 0.04 | 2.92 | 0.22 | 0.2 | 5.29 |
| Mean | 0.31 | 0.15 | 5.40 | 0.03 | 0.02 | 3.57 | 0.15 | 0.04 | 3.31 | 0.21 | 0.18 | 4.99 |
| SD | 0.04 | 0.06 | 0.10 | 0.00 | 0.00 | 0.25 | 0.01 | 0.01 | 0.29 | 0.01 | 0.02 | 0.21 |

Individual patient results tended to reflect the mean changes shown in Tables 6 to 8 with decreases in the proportions of positive cells and increased expression levels at the 24 hr and 72 hr post-infusion time points. There were some exceptions to this pattern. For example, percentages of CD80 and CD86 expressing cells did not decrease as noticeably among CD11⁺ cells as among CD123⁺ cells. In addition, monocytes from CFS patients were similar to those from healthy volunteers in terms of percentages of cells expressing CD86 and in CD86 expression level.

TABLE 6
Kinetics of Maturation Marker Expression in CD123⁺ Dendritic Cells
After Poly(I:C₁₂U) Infusion

| | CD80 | | CD83 | | | |
|---|---|---|---|---|---|---|
| | Positive | | Positive | | CD86 | |
| | Cells | | Cells | | Positive | |
| | (%) | MFI^{b} | (%) | MFI | Cells (%) | MFI |
| Healthy | 0.8 | 55.0 | 11.1 | 78.6 | 35.0 | 71.8 |
| Volunteers^{a} | (1.7) | (33.2) | (7.7) | (69.0) | (17.5) | (25.1) |
| (n = 6) | | | | | | |

Patients
(n = 4)

| | | | | | | |
|---|---|---|---|---|---|---|
| Pre-Infusion | 5.0 | 16.9 | 38.5 | 20.8 | 59.3 | 33.6 |
| | (8.0) | (8.8) | (24.2) | (6.5) | (31.7) | (9.6) |
| 4 hr Post- | 1.8 | 18.5 | 51.5 | 21.5 | 63.8 | 27.8 |
| Infusion | (2.2) | (8.2) | (20.2) | (7.4) | (7.7) | (10.0) |
| 24 hr Post- | 2.3 | 55.3 | 8.1 | 68.5 | 24.8 | 74.3 |
| Infusion | (2.4) | (50.1) | (2.8) | (55.9) | (7.9) | (62.6) |
| 72 hr Post- | 0.3 | 68.5 | 5.1 | 92.7 | 23.1 | 99.3 |
| Infusion | (1.6) | (48.1) | (2.3) | (10.4) | (4.6) | (17.2) |

^{a}Healthy volunteers received no poly(I:C₁₂U);
^{b}MFI, mean fluorescence intensity

TABLE 7
Kinetics of Maturation Marker Expression in CD11⁺ Dendritic Cells
After Poly(I:C₁₂U) Infusion

| | CD80 | | CD83 | | | |
|---|---|---|---|---|---|---|
| | Positive | | Positive | | CD86 | |
| | Cells | | Cells | | Positive | |
| | (%) | MFI^{b} | (%) | MFI | Cells (%) | MFI |
| Healthy | 1.1 | 111.1 | 7.8 | 65.1 | 87.0 | 98.9 |
| Volunteers^{a} | (0.6) | (145.8) | (6.3) | (40.1) | (9.4) | (19.6) |
| (n = 6) | | | | | | |

Patients
(n = 4)

| | | | | | | |
|---|---|---|---|---|---|---|
| Pre-Infusion | 0.4 | 23.5 | 23.4 | 21.3 | 97.1 | 67.4 |
| | (2.0) | (10.7) | (6.7) | (9.0) | (1.8) | (9.4) |
| 4 hr Post- | 3.5 | 17.4 | 19.7 | 18.6 | 97.4 | 66.6 |
| Infusion | (4.7) | (9.4) | (8.8) | (10.2) | (1.6) | (19.8) |
| 24 hr Post- | 3.9 | 46.1 | 10.4 | 81.4 | 67.0 | 98.8 |
| Infusion | (6.7) | (42.1) | (8.0) | (59.7) | (31.5) | (80.3) |
| 72 hr Post- | −0.1 | 115.3 | 2.5 | 98.5 | 82.9 | 101.4 |
| Infusion | (0.5) | (102.5) | (1.4) | (34.3) | (11.4) | (17.6) |

^{a}Healthy volunteers received no poly(I:C₁₂U);
^{b}MFI, mean fluorescence intensity

TABLE 8
Kinetics of Maturation Marker Expression in Monocytes After
Poly(I:C₁₂U) Infusion

| | CD80 | | CD83 | | | |
|---|---|---|---|---|---|---|
| | Positive | | Positive | | CD86 | |
| | Cells | | Cells | | Positive | |
| | (%) | MFI^{b} | (%) | MFI | Cells (%) | MFI |
| Healthy | 1.3 | 86.6 | 11.6 | 80.9 | 66.0 | 119.7 |
| Volunteers^{a} | (0.9) | (30.6) | (8.0) | (31.6) | (27.4) | (30.8) |
| (n = 6) | | | | | | |

Patients
(n = 4)

| | | | | | | |
|---|---|---|---|---|---|---|
| Pre-Infusion | 2.6 | 52.3 | 26.9 | 56.4 | 86.8 | 109.3 |
| | (4.0) | (13.2) | (6.3) | (11.4) | (5.9) | (12.6) |
| 4 hr Post- | 1.7 | 42.5 | 34.5 | 47.9 | 92.0 | 89.8 |
| Infusion | (1.8) | (12.0) | (17.3) | (13.7) | (2.7) | (15.5) |
| 24 hr Post- | 0.9 | 61.4 | 19.5 | 80.9 | 83.2 | 172.2 |
| Infusion | (1.5) | (33.4) | (5.9) | (43.0) | (8.5) | (62.0) |
| 72 hr Post- | 0.0 | 76.1 | 9.9 | 82.5 | 86.7 | 143.9 |
| Infusion | (0.4) | (19.2) | (2.5) | (20.3) | (3.6) | (28.9) |

^{a}Healthy volunteers received no poly(I:C₁₂U);
^{b}MFI mean fluorescence intensity

In summary, use of poly(I:C₁₂U) did not dramatically affect the numbers of CD123⁺ DC, CD11⁺ DC, or monocytes. Following treatment with poly(I:C₁₂U), the patients experienced normalization in the percentages of DC expressing maturation markers and in CD maturation marker expression levels. These trends, particularly the increase in CD maturation markers, were consistently observed in all four patients, revealing a distinct pattern not recognized in cytokine level modulation.

### Cytokine Levels

A random collection of 76 patients were selected for analysis of cytokine and interferon serum levels at baseline, and after 32 weeks of either placebo or poly(I:C₁₂U) treatment. Thirty-six of the 76 patients (47.4%) selected for analyses were placebo patients; 23 of the 36 placebo patients (63.9%) completed the 40-week study. Forty of the 76 patients (52.6%) selected for analyses were poly(I:C₁₂U)-treated patients; 25 of the 40 poly(I:C₁₂U)-treated patients (62.5%) completed the 40-week study. Baseline or pre-study samples were available for all 28 patients who failed to complete the 40-week study (13 placebo and 15 poly(I:C₁₂U)-treated); the last sample collected from these 28 patients was analyzed and reported as the week 32 result. Baseline or pre-study samples were also available for all 48 patients who completed the 40-week study (23 placebo and 25 poly(I:C₁₂U)-treated); the sample collected at week 32, or the last sample available prior to week 32, was analyzed and reported as the week 32 result.

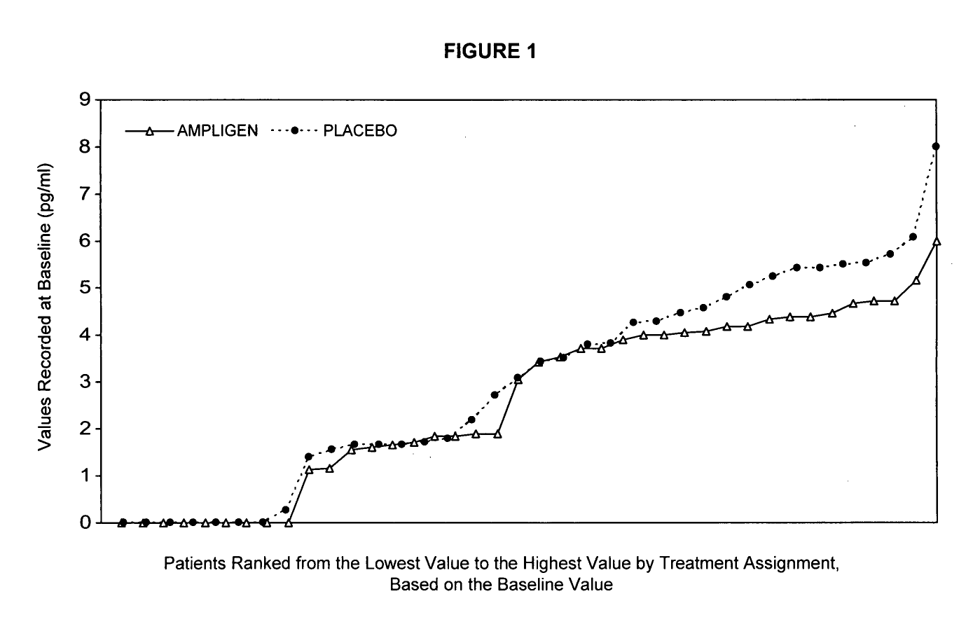

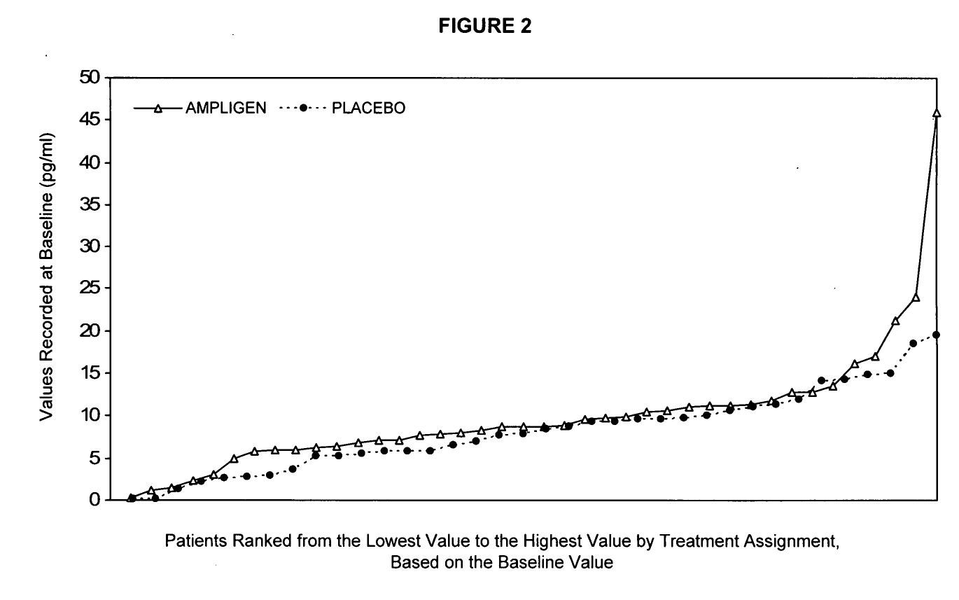

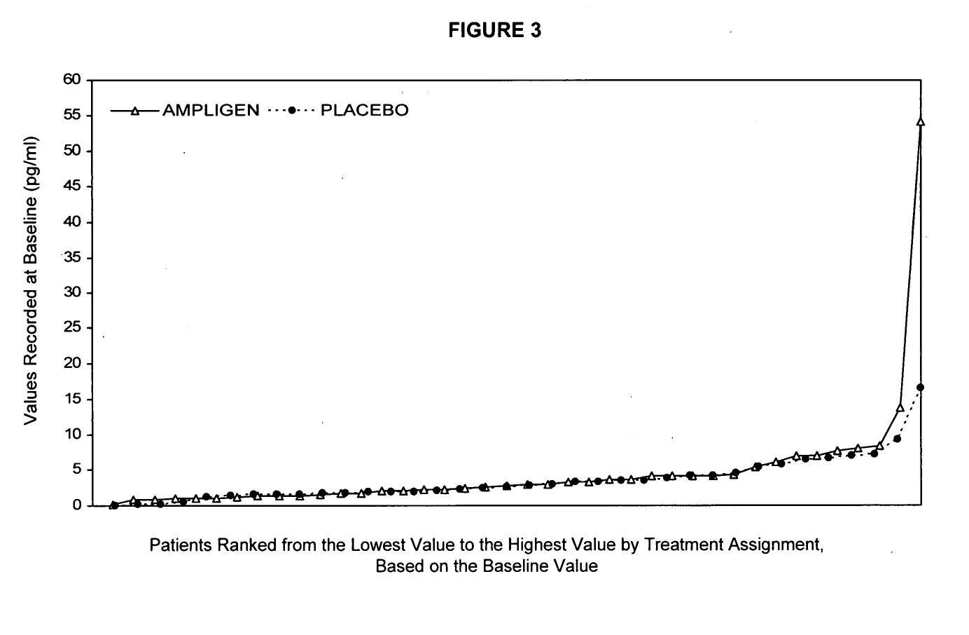

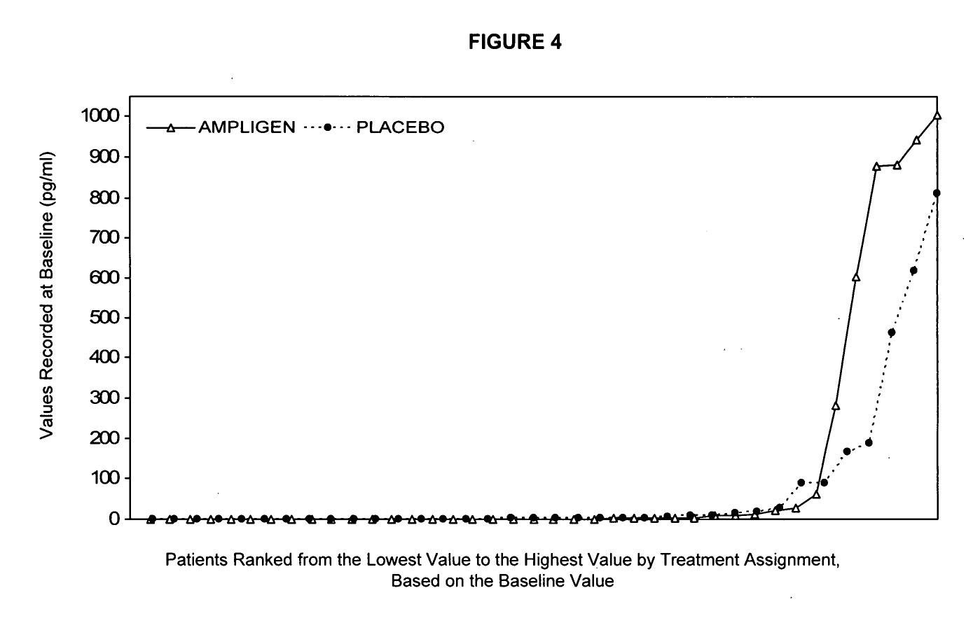

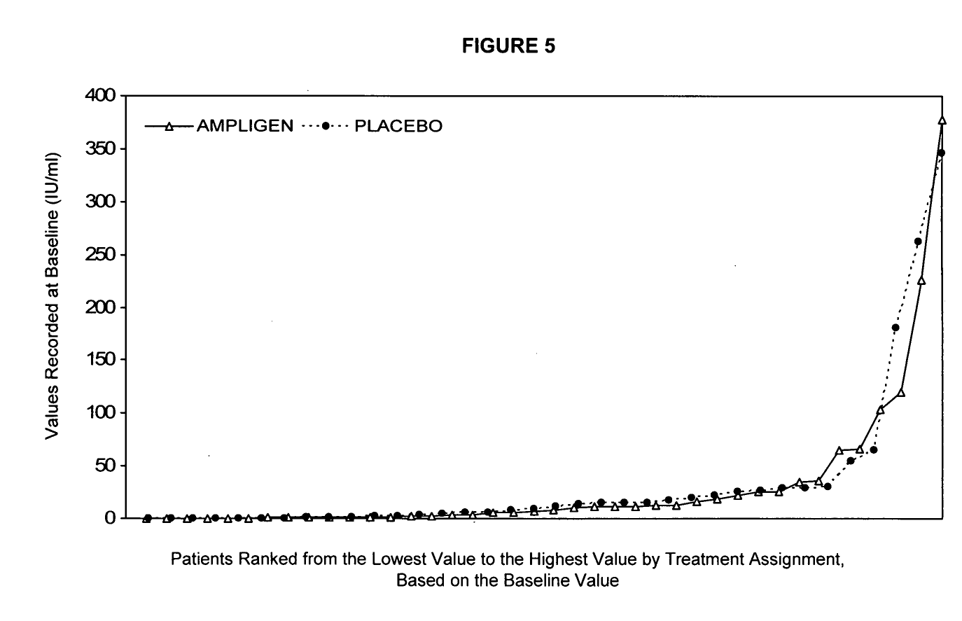

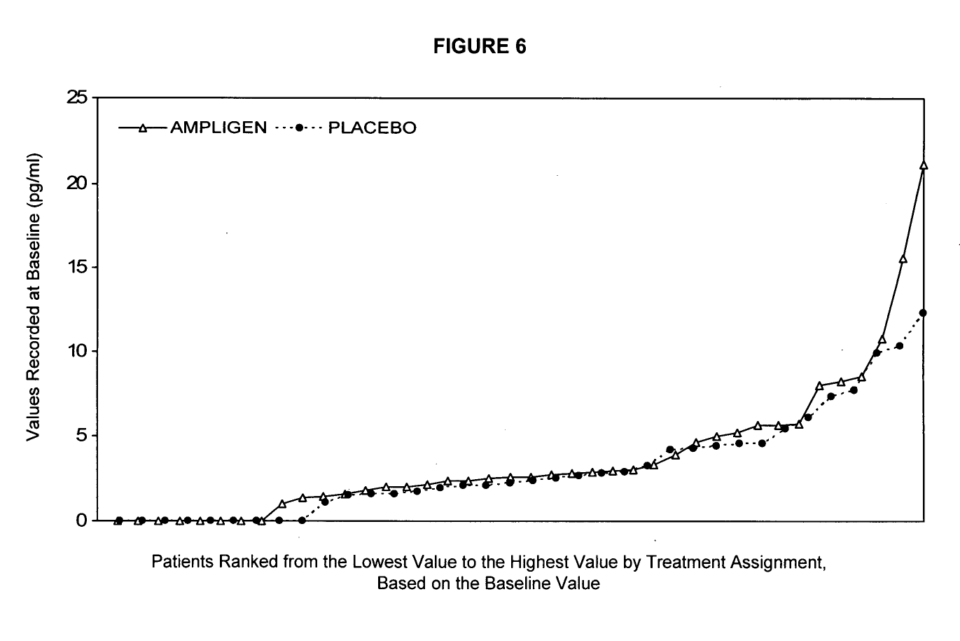

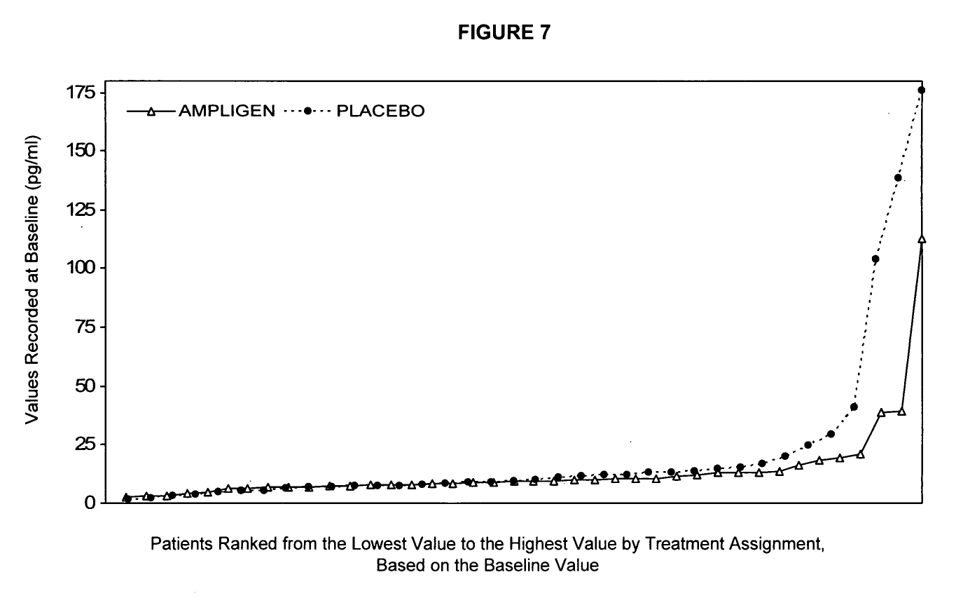

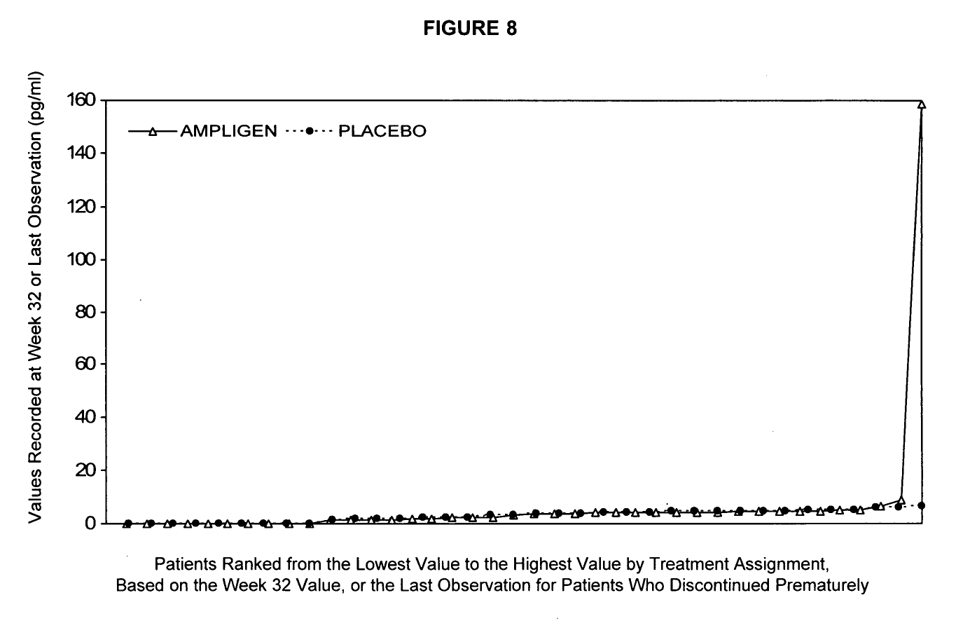

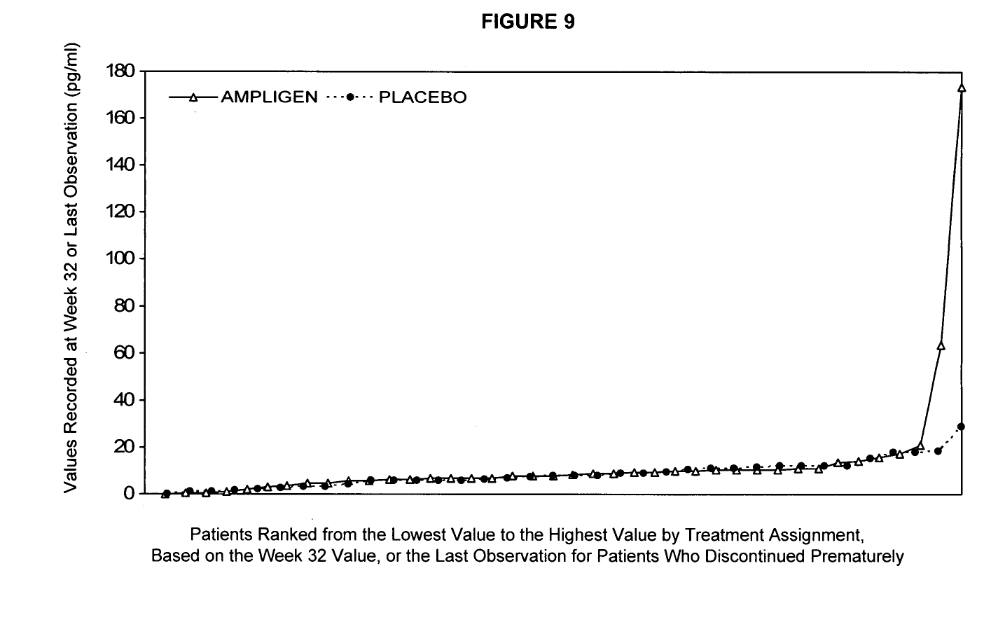

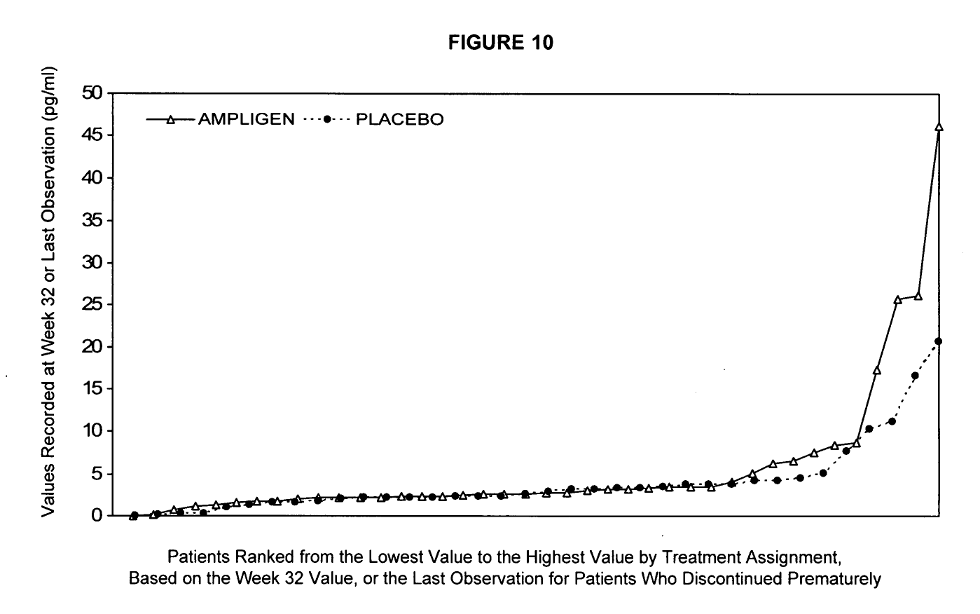

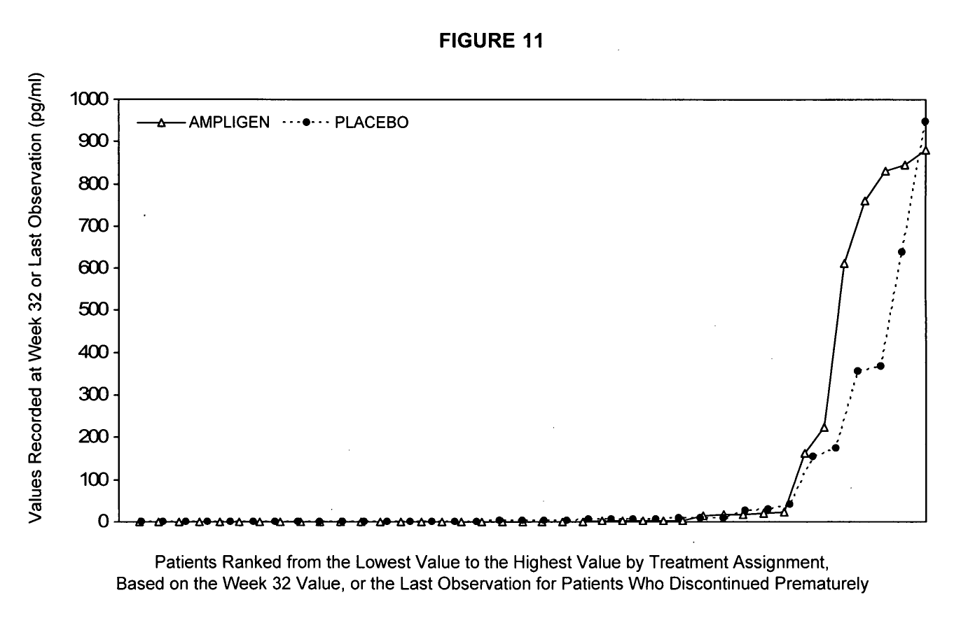

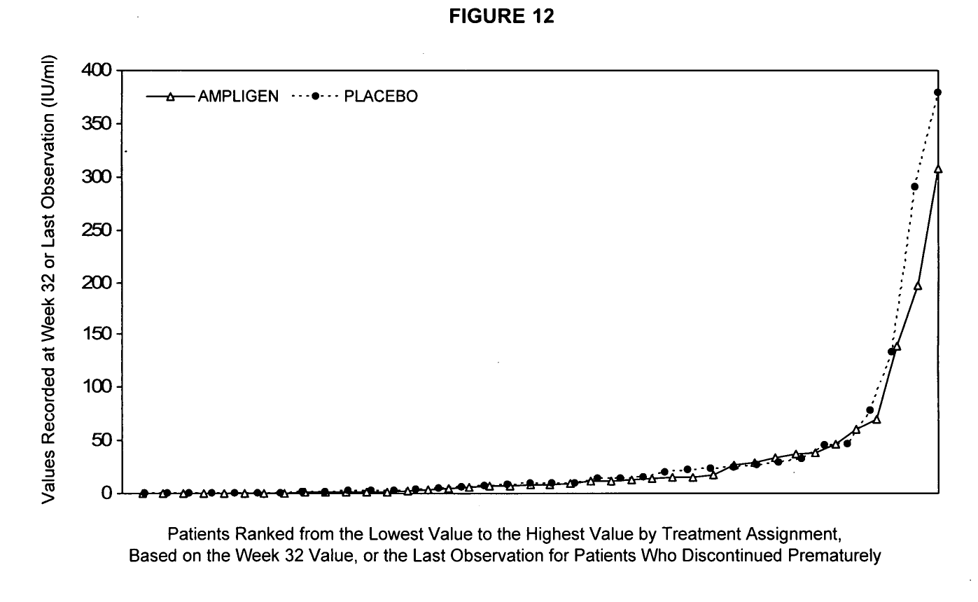

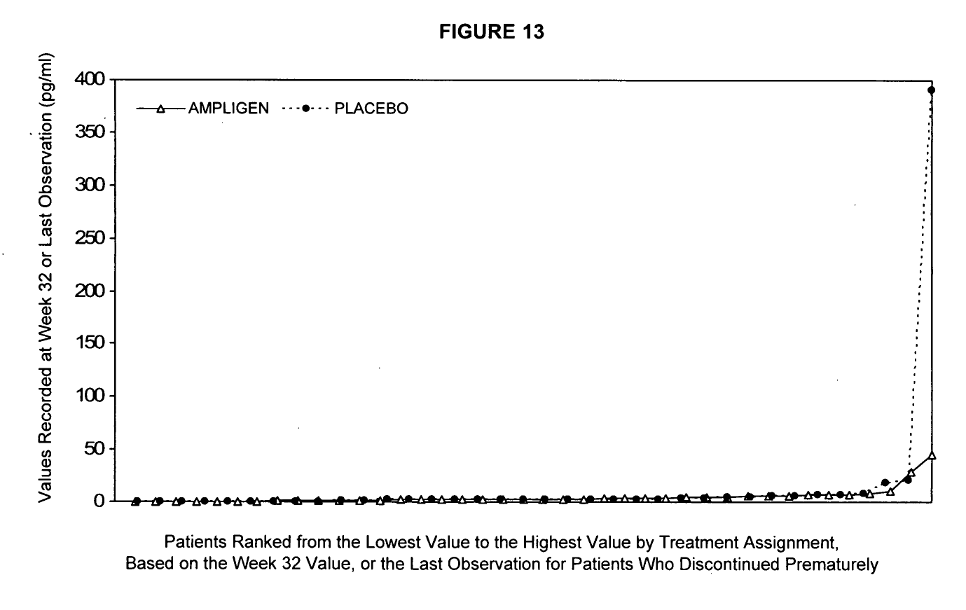

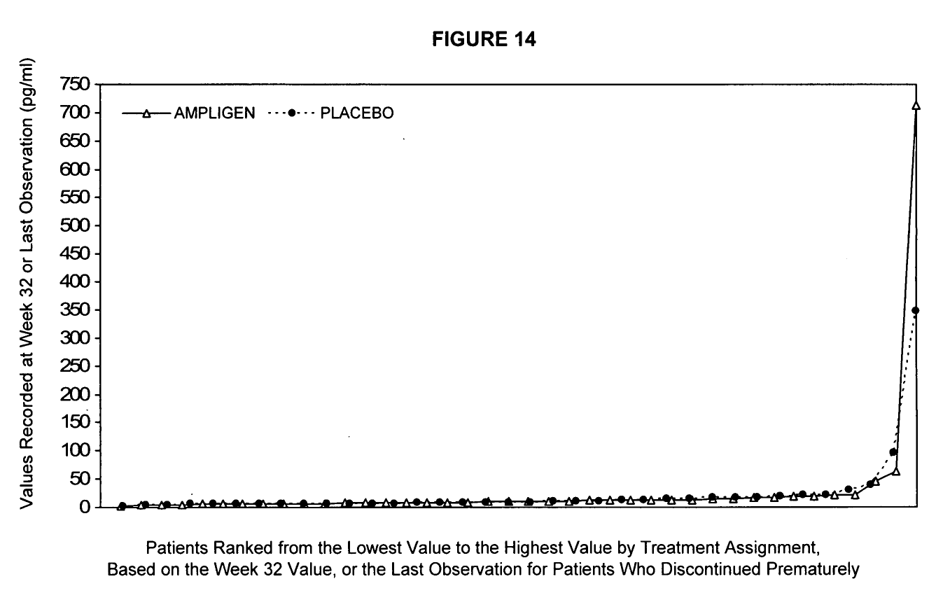

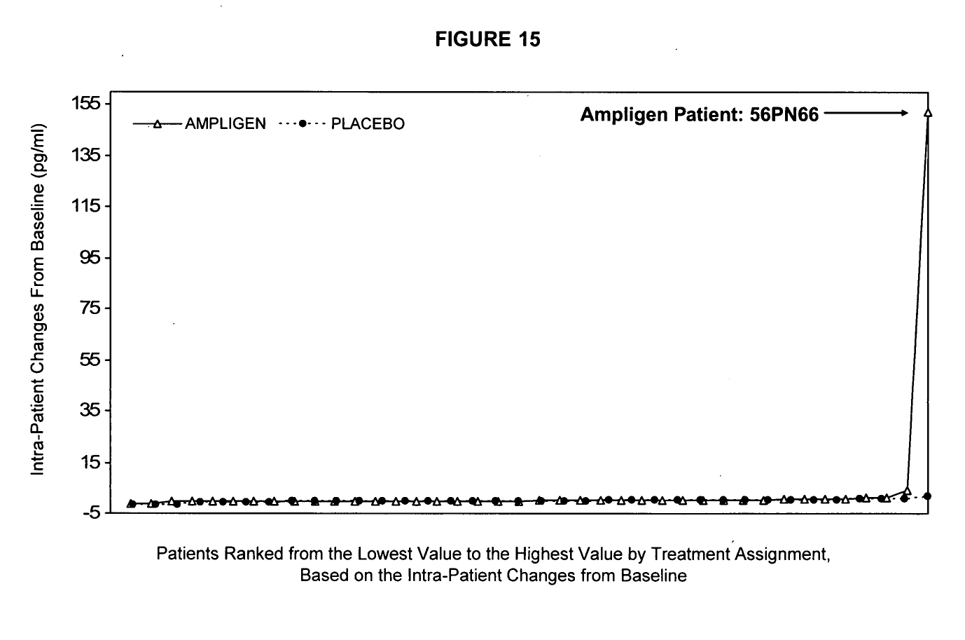

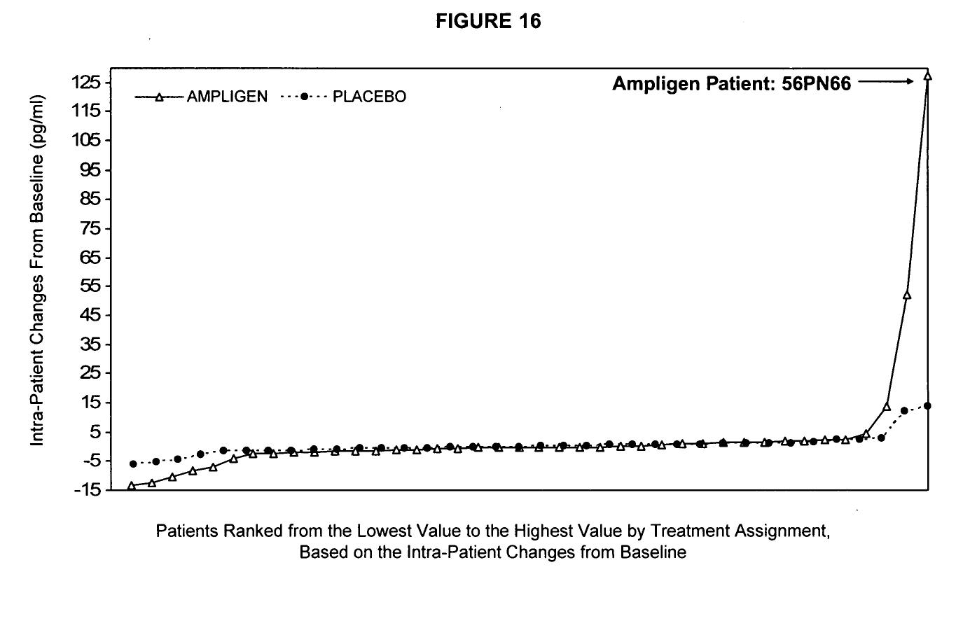

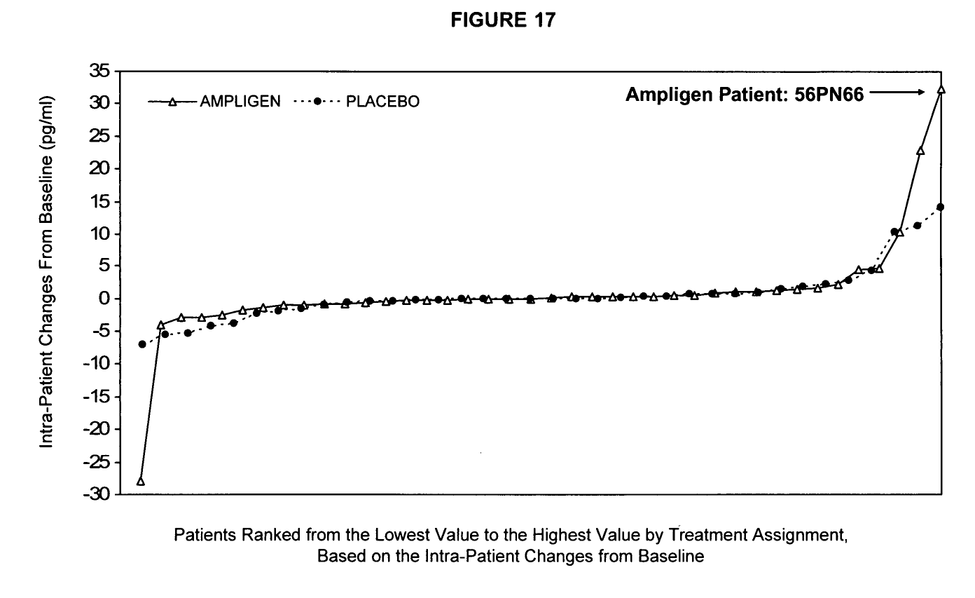

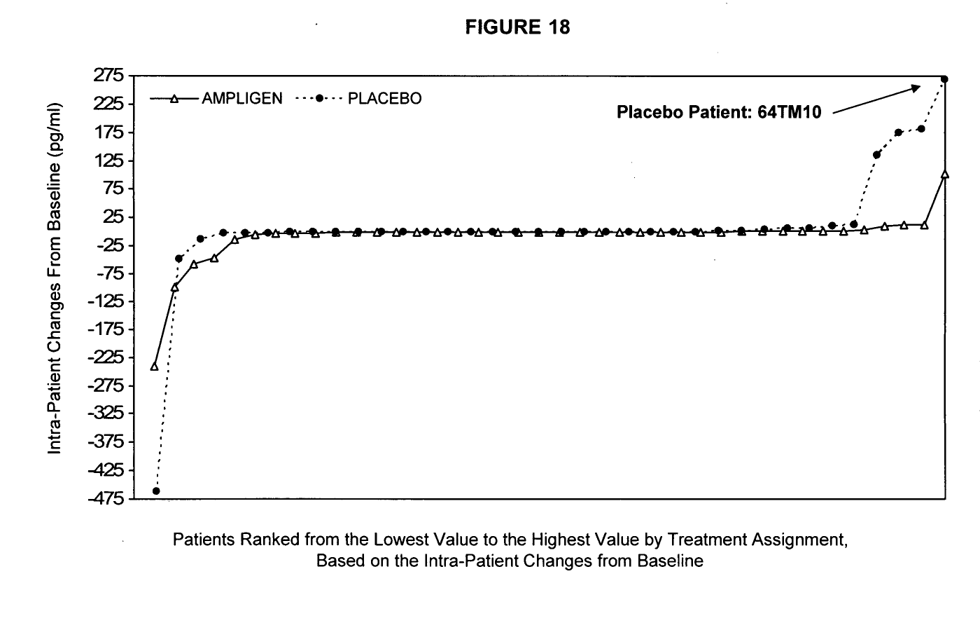

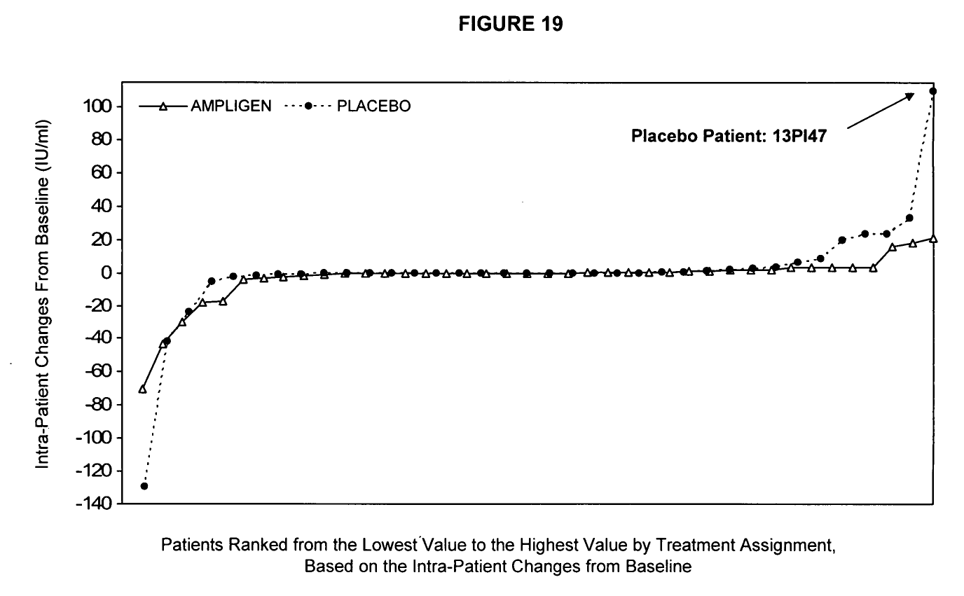

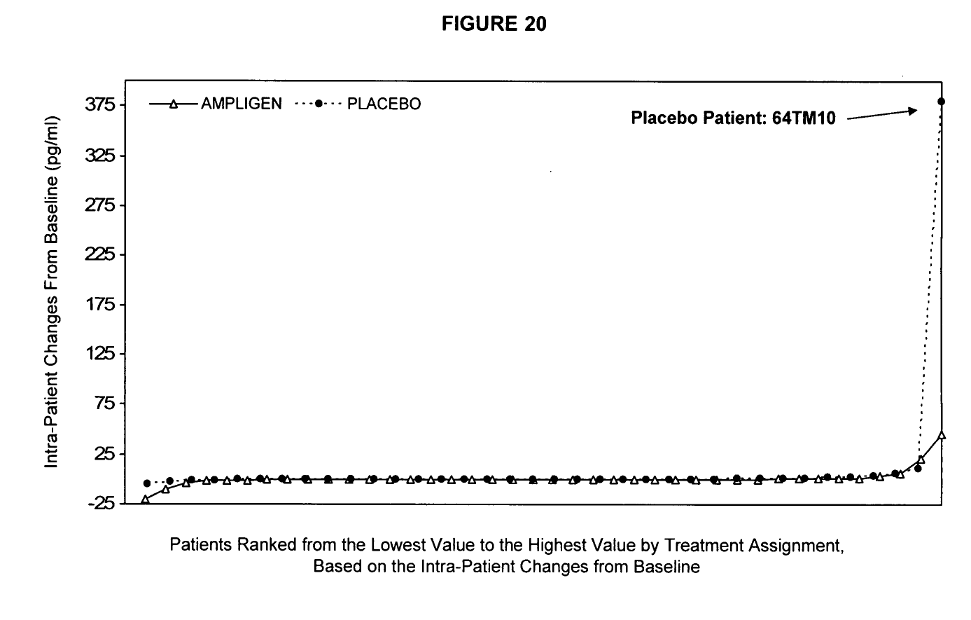

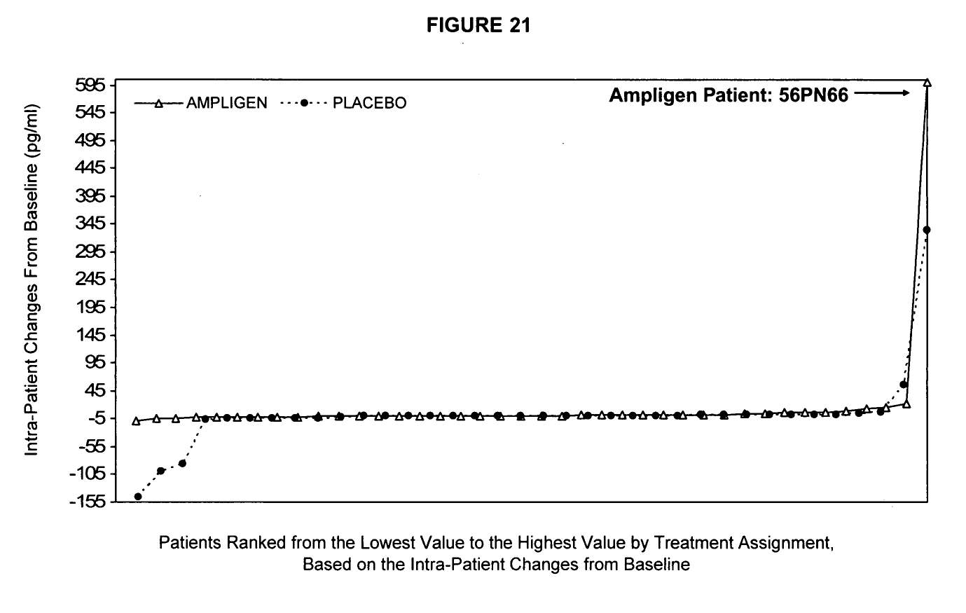

Changes from baseline of interleukin 10 (IL-10), interleukin 12 heterodimer (IL-12) levels, interleukin 6 (IL-6), interferon alpha (IFN-α), interferon beta (IFN-β), interferon gamma (IFN-γ), and tumor necrosis factor alpha (TNF-α) at week 32 are reported in Table 9. FIGS. 1 to 7, which contain the baseline values for all 76 patients, ranked from lowest to highest value by randomized treatment assignment. FIGS. 8 to 14 show the week 32 (or last observation) values. FIGS. 15 to 21 show the difference from baseline at week 32.

No significant modulation of IL-10, IL-12, IL-6, IFN-α, IFN-β, IFN-γ, or TNF-α was seen for the poly(I:C₁₂U) cohort vs. placebo. No difference was observed between poly(I:C₁₂U) and placebo patients in either the interferon or cytokine profiles between patients who completed the study vs. patients who discontinued early. Thus, there was no indication of a cytokine storm with AMPLIGEN® (rintatolimod) treatment.

TABLE 9
Interferon and Cytokine Sera Levels at Baseline and Week 32 by Randomized Treatment Assignment:
AMP-516 Subset Analysis of 76 Patients
One-Factor [Treatment Assignment] Analysis of Variance Tests, Performed on the Ranked Intra-
Patient Changes from Baseline (Last Recorded Value Minus Baseline Value); by Parameter

| | | Independent Variable | Degrees of | Type III Sums | | Probability |
|---|---|---|---|---|---|---|
| Parameter | Dependent Variable | in the Model | Freedom | of Squares | F-Value | Value |
| IL-10 | Last Observation Minus | Treatment Assignment | 1 | 1251.677778 | 2.6667 | 0.107 |
| | Baseline Observation | | | | | |
| IL-12 | Last Observation Minus | Treatment Assignment | 1 | 240.468750 | 0.4898 | 0.486 |
| | Baseline Observation | | | | | |
| IL-6 | Last Observation Minus | Treatment Assignment | 1 | 52.368750 | 0.1061 | 0.746 |
| | Baseline Observation | | | | | |
| INF-α | Last Observation Minus | Treatment Assignment | 1 | 359.218750 | 0.7370 | 0.393 |
| | Baseline Observation | | | | | |
| INF-β | Last Observation Minus | Treatment Assignment | 1 | 95.329861 | 0.1941 | 0.661 |
| | Baseline Observation | | | | | |
| INF-γ | Last Observation Minus | Treatment Assignment | 1 | 747.386111 | 1.5686 | 0.214 |
| | Baseline Observation | | | | | |
| TNF-α | Last Observation Minus | Treatment Assignment | 1 | 1525.277778 | 3.2206 | 0.077 |
| | Baseline Observation | | | | | |

Patents, patent applications, books, and other publications cited herein are incorporated by reference in their entirety.

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability.

All modifications and substitutions that come within the meaning of the claims and the range of their legal equivalents are to be embraced within their scope. A claim which recites “comprising” allows the inclusion of other elements to be within the scope of the claim; the invention is also described by such claims reciting the transitional phrases “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrange-ment of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of treating a patient having chronic fatigue syndrome and impaired physical performance, said method comprising administration to the patient of at least double-stranded ribonucleic acid (dsRNA) or a selective agonist for Toll-like receptor 3 (TLR3) to improve at least one physical symptom of the patient or at least dendritic cell function or phenotype in the patient, with the proviso that the patient does not have chronic cerebral dysfunction.

2. The method according to claim 1, wherein at least a therapeutic amount of TLR3 agonist is administered to the patient.

3. The method according to claim 1, wherein at least a therapeutic amount of dsRNA is administered to the patient.

4. The method according to claim 1, wherein at least a therapeutic amount of mismatched dsRNA is administered to the patient.

5. The method according to claim 4, wherein the mismatched dsRNA comprises poly(I:C₄₋₂₉U).

6. The method according to claim 4, wherein the mismatched dsRNA comprises poly(I:C₁₁₋₁₄U).

7. The method according to claim 4, wherein the mismatched dsRNA comprises poly(I:C₁₂U).

8. The method according to claim 1, wherein at least dsRNA or TLR3 agonist in a therapeutic amount is infused intravenously.

9. The method according to claim 1, wherein at least dsRNA or TLR3 agonist in a therapeutic amount is injected intradermally, subcutaneously, or intramuscularly; inhaled intranasally or intratracheally; or applied intranasally, intratracheal̂, oropharyngeally, or sublingually.

10. Use of one or more different double-stranded ribonucleic acids (dsRNA) or selective agonists for Toll-like receptor 3 (TLR3) in manufacture of a medicament for treatment of a patient having chronic fatigue syndrome and impaired physical performance, with the proviso that the patient does not have chronic cerebral dysfunction.

11. Use according to claim 10, wherein at least one physical symptom of the patient is improved by the treatment.

12. Use according to claim 10, wherein at least dendritic cell function or phenotype in the patient is improved by the treatment.

13. A pharmaceutical composition containing one or more different double-stranded ribonucleic acids (dsRNA) or another selective agonists for Toll-like receptor 3 (TLR3) for treatment of a patient having chronic fatigue syndrome and impaired physical performance, with the proviso that the patient does not have chronic cerebral dysfunction.

14. The composition of claim 13, wherein at least one physical symptom of the patient is improved by the treatment.

15. The composition of claim 13, wherein at least dendritic cell function or phenotype in the patient is improved by the treatment.
